SEQUENCE LISTING (6 sequences)

SEQ ID NO: 1 - DNA, 35 bp, Artificial Sequence
  primer
cccttgaaaggaaaaattctggatatccatcagag

SEQ ID NO: 2 - DNA, 34 bp, Artificial Sequence
  primer
cagaatttttcctttcaagggcctgacacttttc

SEQ ID NO: 3 - DNA, 38 bp, Artificial Sequence
  primer
ggaaaaattctggatatccatcagagttgaatgaaaag

SEQ ID NO: 4 - DNA, 36 bp, Artificial Sequence
  primer
ctctgatggatatccagaatttttcctttcaagggc

SEQ ID NO: 5 - DNA, 31 bp, Artificial Sequence
  primer
gagctcctgaccgcgaaccaccagctgaaag

SEQ ID NO: 6 - DNA, 31 bp, Artificial Sequence
  primer
ctttcagctggtggttcgcggtcaggagctc

(19) United States
(12) Patent Application Publication
(10) Pub. No.: US 2010/0267147 A1
(43) Pub. Date: Oct. 21, 2010

(54) SITE-DIRECTED MUTAGENESIS IN CIRCULAR METHYLATED DNA

(75) Inventor: Miao Qiao, Rockville, MD (US)

Correspondence Address: Miao Qiao;GM Biosciences, Inc, 4539 Metropolitan Ct., Frederick, MD, 21704, US

(73) Assignee: GM Biosciences, Inc., Frederick, MD (US)

(21) Appl. No.: 12/793,622

(22) Filed: Jun. 3, 2010

Related U.S. Application Data
(63) Continuation-in-part of application No. 12/593,289, now abandoned.

(51) Int. Cl. C12N 15/70; C12N 1/21

(52) U.S. Cl. 435/471; 435/252.8

(57) ABSTRACT

Site-specific mutation in methylated circular stranded DNA molecules is conferred by mutagenic primer pairs and methylase deficient Escherichia coli. The mutagenic primer pairs are complementary at 5′ end or 3′ end, or completely complementary to each other. Firstly, mutagenic primer pair is annealed to opposite strands of the methylated circular double-stranded parent DNA molecules. Then, polymerase chain reaction is performed by using unmethylated dNTPs to create unmethylated mutagenized double-stranded daughter DNA molecules. Finally, the reaction mixture of the methylated parent DNA molecules and unmethylated mutagenized daughter DNA molecules is transformed into a methylase deficient E. coli. The replication of methylated parent DNA is inhibited in methylase deficient host cell. In contrast, the unmethylated daughter DNA, which contains the desired mutation, are efficiently replicated in methylase deficient host cell and recovered thereafter. The invention also provides a kit for introducing site-specific mutations in accordance with the described method.

### CROSS-REFERENCE TO RELATED APPLICATIONS

This application is a continuation-in-part of U.S. Ser. No. 12/593,289 which is the US national stage of International Patent Cooperation Treaty (PCT) application Ser. No. PCT/CN2007/001377 filed on Apr. 25, 2007 by the present inventor.

### FEDERALLY SPONSORED RESEARCH

NO

### SEQUENCE LISTING OR PROGRAM

NO

### BACKGROUND

1. Field of the Invention

The present invention relates to the field of molecular biology. Specifically, the present invention provides a novel method for site-specific mutagenesis.

2. Prior Art

Site-directed mutagenesis is a powerful molecular tool for studying the effects of DNA sequence changes on protein function. As a cyclic amplification technique, polymerase chain reaction (PCR) has been widely adopted in site-directed mutagenesis, in which mutagenic primers are used to introduce desired mutations. (see Allemandou et al., J Biomed Biotechnol S, 202-207, (2003); An et al., Appl Microbiol Biotechnol S 68, 774-778, (2005); Hall, et al. Protein Eng. 4:601 (1991); Hemsley, et al. Nucleic Acids Research 17:6545-6551 (1989); Ho, et al. Gene 77:51-59 (1989); Hultman, et al. Nucleic Acids Research 18:5107-5112 (1990); Jones, et al. Nature 344:793-794 (1990); Landt, et al. Gene 96:125-128 (1990); Nassal, et al. Nucleic Acids Research 18:3077-3078 (1990); Nelson, et al. Analytical Biochemistry 180:147-151 (1989); Vallette, et al. Nucleic Acids Research 17:723-733 (1989); Weiner, et al. Gene 126:35-41 (1993), which are all incorporated herein in their entity by reference).

Based on this methodology, after amplification by PCR, selection of mutated DNA and removal of parental plasmid DNA has become a key step and can be accomplished by various ways. Currently, the most popular selection methods include: 1) replacement of dCTP by hydroxymethylated-dCTP during PCR, followed by digestion with restriction enzymes to remove non-hydroxymethylated parent DNA; 2) simultaneous mutagenesis of both the studied gene and an antibiotic resistance gene which results in a different antibiotic resistance, the new antibiotic resistance facilitating the selection of the desired mutation thereafter; 3) using methylated DNA as template in PCR, then digesting the parent methylated template DNA by restriction enzyme DpnI which cleaves only methylated DNA, by which the mutagenized unmethylated DNA are recovered; and 4) circularization of the mutated PCR products in an additional ligation reaction to increase the transformation efficiency of mutated DNA. Description of exemplary methods can be found in e.g. U.S. Pat. Nos. 7,132,265, 6,713,285, 6,673,610, 6,391,548, 5,789,166, 5,780,270, 5,354,670 and 5,071,743, all of which are incorporated herein in their entity by reference. In addition, other in vivo and in vitro methods have also been developed. For example, in non-amplification based in vivo site-directed mutagenesis methods, the incorporation of dUTP into parental DNA during growth of the vector can be selected against in dut⁺, ung⁺ Escherichia coli (E. coli) cells (see Kunkel Proc. Natl. Acad. Sci. (U.S.A.) 82:488-492 (1985)). Other in vitro methods for selection of the mutated strand include: 1) unique restriction site elimination (see Deng, et al. Analytical Biochemistry 200:81-88 (1992)); 2), solid phase techniques (where the parental DNA remains attached to the solid phase; see Hultman, et al. Nucleic Acids Research 18:5107-5112 (1990); Weiner, et al. Gene 126:35-41 (1993)); and 3) incorporation of modified bases in the newly replicated DNA (Taylor et al. Nucleic Acids Research 13:8765-8785 (1985); Vandeyar, et al. Gene 65:129-133 (1988)). All cited references are incorporated herein by reference.

In all mutagenesis methods mentioned above, transformation into E. coli is the way to amplify plasmids and is generally the last step. However, to facilitate the selection of mutant DNA, all these methods have to include additional steps before or after the PCR amplification, such as in vitro enzyme digestion. Given the different methods of site-directed mutagenesis that are currently in use, they all need more steps to accomplish the selection against parental DNA and thus need more time and efforts.

### SUMMARY OF THE INVENTION

In order to provide researchers with useful methods of site-directed mutagenesis for saving time and efforts and increasing the efficiency, the present invention provides a novel method for selection of the mutated DNA and removal of the parental plasmid DNA. The method of the invention is simpler than any current methods and can be used to generate variant mutations such as substitution, insertion and deletion more efficiently.

Not bound by any theory, the present invention is mainly based on the observation that methylated DNA has a very poor transformation frequency into methylase deficient E. coli (such as Dam and Dcm deficient (Dam⁻Dcm⁻) E. coli), as the result of the poor replication efficiency. On the contrary, unmethylated DNA has very high transformation frequency into methylase deficient E. coli, as the result of a very high replication efficiency.

Therefore, in the first aspect, the present invention provides a facile and effective method for efficiently introducing specific site-directed mutations of interest into a target circular methylated nucleic acid (FIG. 1), comprising:
(a) performing polymerase chain reaction (PCR) using DNA polymerase(s), complementary mutagenic primers, unmethylated dNTPs and a circular methylated nucleic acid to be mutagenized as PCR template;
(b) transforming the mixture of PCR products from step (a) into a methylase deficient E. coli strain, in which the mutagenized unmethylated nucleic acid is efficiently replicated; and
(c) recovering the mutagenized unmethylated nucleic acids from the E. coli strain.

In embodiments of the invention, partially complementary or completely complementary primers selected as the mutagenic primer pairs and containing desired mutation(s) such as substitution, insertion or deletion, with respect to the target DNA sequence, can be used to carry out the site-directed mutagenesis. The mutation is located in the complementary or non-complementary region of the primers.

In the methods of the invention, circular methylated parent DNA molecule to be mutagenized is used as template for polymerase chain reaction (PCR) with the mutagenic primer pairs. PCR is performed by cycles of denaturation, annealing and extension by using unmethylated dNTPs. The PCR product, which is a mixture of methylated parent template and unmethylated mutagenized daughter DNA, is transformed into DNA methylase deficient host cells. Preferentially the methylase deficient host cells are Dam⁻Dcm⁻. E. coli cells. The methylated parent DNA replicates poorly in the methylase deficient E. coli. In contrast, the unmethylated daughter DNA gets replicated very efficiently in methylase deficient E. coli and gets recovered thereafter.

The methylase deficient cells in the method of the present invention can be any of a variety of kinds of methylase deficient cells. Preferentially, the methylases are Dam and Dcm.

Therefore, in one embodiment of the present invention, the methylase deficiency in methylase deficient E. coli is transient. In another embodiment, the methylase deficiency in methylase deficient E. coli is permanent.

In yet another embodiment, the methylase deficiency in methylase deficient E. coli is inducible. In still another embodiment, the methylase deficiency in methylase deficient E. coli is non-inducible.

In a more preferred embodiment of the invention, the methylase deficient cells are Dam and Dcm deficient E. coli and the deficiencies are non-inducible and permanent.

In the most preferable embodiment, the methylase deficient E. coli strain used in the method of the present invention is the strain ER2925 or SK383.

In one embodiment of the present invention, said circular methylated nucleic acid is methylated in vitro.

In another embodiment, said circular methylated nucleic acid is methylated in vivo.

In a further embodiment of the present invention, in step (a) the primers are complementary at their 5′ end and/or 3′ end.

In another embodiment, in step (a) the primers are completely complementary to each other.

In a preferred embodiment of the present invention, in step (a) said DNA polymerase is temperature stable.

Another aspect of the invention is to provide a kit introducing mutation(s) into a selected DNA molecule for mutagenesis, said kit comprising, but not limited to, methylase deficient cells, preferentially methylase deficient E. coli cells.

Further aims, objects, and advantages of the mutagenesis protocol described and claimed herein will become apparent upon a complete examination of the Detailed Description, attached Claims, and accompanying drawing figures.

### BRIEF DESCRIPTION OF THE DRAWINGS

FIG. 1. A schematic outline of mutagenesis strategy

Step (a) shows that parent methylated plasmid was used as template for mutagenesis. The dash line represents methylated chains. Step (b) shows that two complementary primers introducing desired mutation anneal to opposite strands of the template DNA, respectively. The symbol of cross represents the desired mutations. Then, daughter unmethylated chains with mutations are synthesized by polymerase chain reaction (PCR). The bold line represents unmethylated chains. Step (c) shows that the product of PCR contains double stranded DNAs which are methylated or unmethylated or hemi-methylated. Step (d) shows that the outcome of transformation of PCR product into a methylase deficient E. coli. The replication of both methylated and hemi-methylated DNA is inhibited, whereas only unmethylated mutagenized daughter DNA replicate efficiently and enriched thereafter.

### DETAILED DESCRIPTION OF THE INVENTION

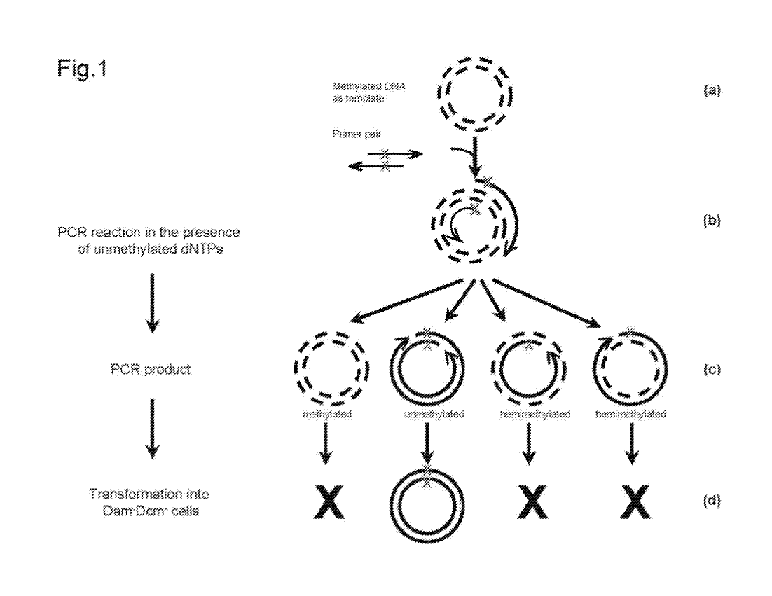

The present invention provides a method for efficiently introducing specific site-directed mutations into a target circular methylated nucleic acid as illustrated in FIG. 1, comprising:
(a) performing polymerase chain reaction (PCR) using DNA polymerase(s), complementary mutagenic primers, unmethylated dNTPs and the selected circular methylated nucleic acid to be mutagenized as PCR template;
(b) transforming the mixture of PCR product from step (a) into a methylase deficient E. coli strain, in which the mutagenized unmethylated nucleic acid is efficiently replicated; and
(c) recovering the mutagenized unmethylated nucleic acids from the E. coli strain.

The term “methylase”, as used herein, refers to DNA methyltransferases (MTases), which for example transfer methyl group from S-adenosylmethionine to either adenine or cytosine residues. Methylases are found in a wide variety of prokaryotes and eukaryotes. Methylation needs to be considered when digesting DNA with restriction endonucleases because cleavage can be blocked or impaired when a particular base in the recognition site is methylated.

In prokaryotes, methylases have most often been identified as elements of restriction/modification systems that act to protect host DNA from cleavage by the corresponding restriction endonucleases. Most laboratory strains of E. coli contain three site-specific DNA methylases. The methylase encoded by the dam gene (Dam methylase) transfers a methyl group to the N⁶ position of the adenine residues in the sequence GATC (Marinus, et al. J. Bacteriol. 114, 1143-1150 (1973); Geier, et al. J. Biol. Chem. 254, 1408-1413 (1979)). The Dcm methylase, encoded by the dcm gene, methylates the internal cytosine residues in the sequences CCAGG and CCTGG (Marinus, et al. J. Bacteriol. 114, 1143-1150 (1973); May, et al. J. Bacteriol. 123, 768-770 (1975)) at the C5 position. The EcoKI methylase, M. EcoKI, modifies adenine residues in the sequences AAC(N6)GTGC and GCAC(N6)GTT. Some or all of the sites for a restriction endonuclease may be resistant to cleavage when isolated from strains expressing the Dam or Dcm methylases if the methylase recognition site overlaps the endonuclease recognition site.

Almost all strains commonly used in cloning express Dam and Dcm (Dam⁺Dcm⁺), and many are M⁺ EcoKI.

As used herein, “site-directed mutagenesis” refers to a process in which a mutation is created at a defined site in a DNA molecule. The defined site refers to sites chosen as desired according to need of research. The DNA molecule for mutagenesis usually is a circular molecule known as a plasmid. In general, site-directed mutagenesis requires that the wild-type gene sequence be known. This technique is also known as “site-specific mutagenesis” or “oligonucleotide-directed mutagenesis”.

Consequently, “site-directed mutation” means mutations created at a defined site in a DNA molecule by technique of site-directed mutagenesis. The site of mutagenesis is chosen as desired according to different needs.

As used herein, “methylase deficient E. coli” means DNA methylase is functionally deficient in E. coli. The functional deficiency of DNA methylase results from lower/absent transcription of DNA methylase gene in RNA level by variety of means, or from lower/absent activity of methylase in protein level by variety of means, or both. The methylase deficiency is inducible or non-inducible, transient or permanent, or every possible combination (see below). DNA synthesized in methylase deficient E. coli is methylated non-efficiently compared to that in methylase non-deficient E. coli.

In every embodiment of the invention, the method of the present invention involves using methylase deficient E. coli as a selection tool to efficiently eliminate methylated parent DNA whereas unmethylated mutagenized DNA is specifically enriched. The method requires minimum effort to obtain the desired mutagenesis, thereby it decreases time and cost. This invention has an advantageous combination of features: (1) broad compatibility (2) high mutagenesis efficiency, and (3) simplicity.

In every embodiment of the invention, the method of the invention also relates to using methylated DNA as the target of mutagenesis. In some embodiments, the methylated DNA of the present invention is achieved in vitro. In some other embodiments, the methylated DNA is achieved in vivo. Still in some other embodiments, the methylated DNA is a combination of DNA molecules achieved in vitro and in vivo. The in vitro methylation of DNA can be for example achieved simply by either an in vitro methylase reaction or incorporation of methylated dNTPs during in vitro synthesis. The in vivo methylation of DNA can be for example easily achieved by replication of the DNA in eukaryotic or prokaryotic cells, preferably in E. coli cells, that endogenously express a suitable methylase. In some embodiments of the present invention, common laboratory strains of E. coli such as, but not limited to, DH5a, Top10, XL1-Blue containing DNA methylases Dam and Dcm (Dam⁺Dcm⁺), were used to generate methylated DNAs. As known by persons skilled in art, methylases Dam and Dcm are the two major DNA methylases in prokaryotes. They have most often been identified as elements of restriction/modification systems that act to protect host DNA from cleavage by the corresponding restriction endonucleases. Also as known by the art, DNA of several Kb in length generally contains a large amount of methylation sites of Dam (˜1 site per 256 bp) or Dcm (˜1 site per 512 bp), wherein many of these were methylated by Dam and Dcm in E. coli. In addition, in some embodiments, the DNA sequences targeted for mutagenesis in the method of the invention are double-stranded; in some other embodiments, the DNA sequences targeted for mutagenesis in the method of the invention are single-stranded.

In some embodiments, mutations resulted from the present invention have not only one or more substitutions in the DNA sequences of interest, but also either insertion or deletion. In some other embodiments, mutations may be more complicated, which include more than one type. In some extreme embodiments, mutations include all the three types: substitution, insertion and deletion. Two or more primers are employed for mutagenesis by the present invention. Preferably, two primers are employed by the present invention. The two primers are partially complementary at the 5′ end and/or 3′ end or completely complementary to each other. Mutations are included in one or both of the primers, and located in either the complementary region or the non-complementary region, or in both the complementary and non-complementary. In one preferred embodiment of the invention, if two partially complementary primers are employed, the mutations are located in the complementary region of both two primers. In another preferred embodiment of the invention, if two partially complementary primers are employed, the mutations are located in the non-complementary region of one or both primers. In another preferred embodiment of the invention, if two completely complementary primers are employed, the mutations are located in the middle of both primers.

The primers can be chemically synthesized which are commercially available. Preferably, the primers are synthesized by using unmethylated dNTPs. The requirement of the primers for the method of the subject invention such as purification is identical to that of primers for conventional PCR, which is well known by the skilled in the art. Generally, primer purification by desalting column is good enough. 5′ end phosphorylation of primers is not necessary.

In the embodiments of the present invention, the length of the primers typically ranges from 20 nt to 50 nt, preferably from 25 nt to 45 nt. In case of insertion, the primers may be longer than 50 nt. The complementary region of the primers typically ranges from 10 nt to 50 nt. The method for designing primer pairs is well known by persons skilled in the art.

In every embodiment, the initial step of the method of the present invention is generally to hybridize the mutagenic primers to a target nucleic acid strand. In order to anneal the mutagenic primers to the DNA sequences of interest, a sufficient denaturation step is required. A sufficient denaturation of template methylated DNA is easily achieved by, for example, heating or chemicals. Preferably, the template methylated DNA is heated at 94° C. to 98° C. for 30 min or less. As known by persons skilled in the art, the sufficient denaturation serves for two purposes: 1) completely denaturing the double-stranded template, which makes annealing of primers to the opposite chain of templates more sufficiently; 2) decreasing the transformation efficiency of the template DNA, which ultimately increases the mutagenesis rate of the method of the invention.

Following the denaturation, PCR is performed by using unmethylated dNTPs. In all embodiments of the present invention, after the annealing of the mutagenic primers to the opposite strand of the methylated DNA template, the primers extend and synthesize two novel daughter chains with the mutation incorporated. A hybrid hemimethylated double strand DNA forms, which comprises one methylated template chain and one unmethylated daughter chain. Unmethylated dNTPs are used in this invention to ensure the daughter chains are unmethylated. In the following cycles of PCR, a) the primers may anneal to the opposite daughter chains synthesized in previous cycles and double stranded DNA molecules are synthesized and amplified thereafter; and/or b) the two opposite daughter chains may anneal to each other to directly form double stranded DNA molecules. In the embodiment that two primers partially complementary at their 5′ end are employed, mutagenized double stranded DNA molecules are from both a) and b). In the embodiment that two primers completely complementary to each other, mutagenized double stranded DNA molecules are only from b). In this case, the yield of PCR does not exponentially increase because one daughter chain cannot be used as template by the opposite primer in the following cycles. Preferably the number of PCR cycles are 30 cycles or less, more preferably 20 cycles or less are performed. Generally more PCR cycles are required for complex mutations such as long nucleotide substitution and/or insertion and/or deletion. The optimal numbers for PCR cycles need to be determined individually in different cases according to the template concentration, primer annealing temperature, salt concentration, and amplification efficiency of the DNA polymerase. The minimal cycle number is chosen as long as enough unmethylated mutagenized double stranded DNA molecules are synthesized, in order to minimize the chance of spontaneous mutations introduced by DNA polymerase.

In the embodiments of the present invention, DNA polymerases employed in the present invention are either thermostable or non-thermostable. In a preferred embodiment, the DNA polymerase is a thermostable polymerase. In a more preferred embodiment, the DNA polymerase is a high-fidelity polymerase in order to decrease spontaneous mutations during amplification. Exemplary DNA polymerases compatible to the present invention include, but not limited to, Taq polymerase, pfu polymerase, pfx polymerase, KOD polymerase, and the like. In some embodiments, a mixture of different DNA polymerases is used. The mixture of different DNA polymerases may be different kinds of polymerases or may be the same kind but containing wild-type polymerases and the functional mutants. For example, in an embodiment, a mixture of polymerases contains two different polymerases wherein one has 5′-3′ exonuclease activity and the other does not have. In another embodiment, a mixture of polymerases contains both wild-type and a mutant of the same kind of polymerase wherein one has 5′-3′ exonuclease activity and the other does not have. Description of how to use mixture of polymerases can be found for example in U.S. Pat. No. 5,436,149; Cheng et al., Proc. Natl. Aca. Sci. USA 91:5695-9 (1994), which are incorporated herein by reference.

In the embodiments of the present invention, the PCR product is a mixture containing a variety of double stranded DNAs, including the molecules that I) both strands are methylated parent DNA (unmutagenized), 2) one strand is methylated parent DNA (unmutagenized) and the other strand is unmethylated daughter DNA (mutagenized), 3) both strands are unmethylated daughter DNA (mutagenized). Without any treatment such as purification, digestion and so on, the crude mixture of PCR product is directly transformed into competent cells in order to enrich and recover mutagenized DNA. In the embodiments, cells, preferentially E. coli cells, employed by the method of the subject invention for transformation are methylase deficient. In methylase deficient cells, both methylated and hemi-methylated DNA molecules fail to replicate efficiently, which further causes poor transformation efficiency, whereas unmethylated DNA molecules replicate very efficiently and have high transformation efficiency. It has been shown that replication initiation is suppressed when plasmid DNA is hemimethylated at methylation sites (Russell, D. W. and Zinder, N. D. (1987) Cell 50, 1071-1079). Therefore, after the 1st round of replication in methylase deficient E. coli cells, methylated DNA becomes hemi-methylated DNA and its further replication is inhibited. By transforming into methylase deficient cells, only DNA of 3) is replicated and enriched whereas DNA replication of 1) and 2) is inhibited.

To distinguish the cells containing DNA that both strands are unmethylated daughter DNA (desired DNA) from the cells containing DNA that neither both strands are unmethylated or only one strand is unmethylated daughter DNA (undesired DNA), in the embodiments of the present invention, a selection marker is concatenated with the gene to be mutagenized in the template DNA. In some preferred embodiments, a selection marker is an antibiotic-resistance gene such as ampicillin-resistance, kanamycin-resistance, tetracycline-resistance, chloramphenicol-resistance gene, and the like. The antibiotic-resistance genes are synthesized intactly in the daughter DNA during PCR reaction. Following the transformation into methylase deficient E. coli, the failed replication of the undesired DNA deprives the antibiotic resistance feature of the recipient E. coli, therefore these E. coli cells containing undesired DNA do not form a colony in the presence or corresponding antibiotics. In contrast, only cells containing the desired DNA can form a colony because the desired DNA replicates efficiently and the antibiotic resistance feature is passed to the next generation. In the method of present invention, transformation functions not only in transformation itself but also in selection and enrichment of mutagenized DNA.

Methylases in the E. coli include, but not limited to, dam methylase, dcm methylase, and the like. One or more kinds of methylases may be deficient simultaneously in cells. In one preferable embodiment of the present invention, both Dam and Dcm are deficient (Dam⁻Dcm⁻). Examples of suitable Dam⁻Dcm⁻ E. coli strains include, but not limited to, ER2925, SK383, JM110, and GM1915, etc.

In some embodiments of the present invention, the methylase deficiency is either permanent or transient. The permanent methylase deficiency can be readily achieved by disruption of the genome of E. coli by means of such as, but not limited to, deletion and/or knockout of methylase genes, substitution and/or insertion of a piece of nucleotides in/around the methylase genes which lead to the suppression of expression or frame shift of the methylase genes, constitutive expression of an inhibitor of methylases which inhibits activity of methylases, constitutive expression of a specific protease of methylase which degrades methylases, constitutive expression of an antibody of methylases which neutralize the activity of methylases, etc. The transient deficiency means that the deficiency only occurs in a certain period of time. The transient methylase deficiency can be readily achieved by, but not limited to, the transient suppression of transcription or translation of methylases, the transient inactivation of methylase by means of a transient process such as rapid degradation or saturation or neutralization or compartmentation or aggregation. The duration of the transient deficiency should be long enough to enrich the desired DNA whereas inhibiting the undesired DNA.

In some other embodiments of the present invention, the methylase deficiency is non-inducible or inducible. In the embodiments relating to inducible methylase deficiency, the methylase deficiency is induced by any means such as, but not limited to, chemicals, drugs, expression of proteins, and the like. In some embodiments, the methylase deficiency is the combination of non-inducible, inducible, permanent and transient. Therefore in the embodiments of the invention, the methylase deficiency may be non-inducible permanent, inducible permanent, non-inducible transient and inducible transient. Currently widely used Dam⁻Dcm⁻ E. coli strains such as ER2925, SK383 are examples of non-inducible permanent methylase deficiency.

The previous description mainly concerns about using double-stranded DNA as target of mutagenesis. A person of ordinary skill of art may easily extend the method of the subject invention to introduce mutagenesis for single-stranded circular methylated DNA. In the embodiments where single stranded circular DNA is used as the target of mutagenesis, the same two partially complementary or completely complementary primers as described above are employed and the same procedure is followed.

Another aspect of the invention is to provide a kit for performing site-directed mutagenesis. The kit may contain necessary reagents and instructions to perform the subject invention. In one embodiment, the kit of the invention at least contains: methylase deficient E. coli cells, control primers, and control templates. In another embodiment, the kit of the invention may contain: methylase deficient E. coli competent cells, DNA polymerase, nucleotide triphosphates, methylase, concentrated reaction buffers, and the like. A preferred kit of the invention comprises a DNA polymerase, methylase deficient E. coli competent cells, control primers, and control templates, nucleotide triphosphates, concentrated reaction buffers.

One of the advantages of the method of this invention is simplicity. No separate selection steps after generation of the mutagenized chains are required, which on the contrary is the most crucial step employed by most current available methods by variety of means such as specific digestion of parent unmutagenized DNA. In this method of the present invention, transformation into host cell functions as both selection and recover steps. The different status of methylation between parent DNA and mutagenized daughter DNA is distinguished by methylase deficient cells based on transformation frequency and ability to replicate. The present invention utilizes methylase deficient cells as a selective tool to efficiently eliminate methylated parent DNA whereas unmethylated mutagenized daughter DNA is specifically enriched. The step of selection and the step of the recovery thereafter are integrated into a single step of transformation, which confer the most distinct advantage of the present invention.

Another advantage of the present invention is broad compatibility with most current cloning systems. In most cases in laboratory world-widely, DNA molecules are replicated in cells endogenously expressing methylase. Thus these DNA molecules are suitable to serve as the template in the present invention without any treatment such as in vitro methylation reaction. Most cells are appropriate for the in vivo methylation of circular DNA to be mutagenized, wherein E. coli cells are preferred. On the other hand, circular methylated DNA to be mutagenized may also be methylated in a cell free system in an in vitro methylation reaction by methylases.

### EXAMPLES

The following examples are included herein solely to provide a clearer understanding of the subject invention, which do not intend to limit the scope of the subject invention in any manner whatsoever.

The following protocols provide procedure to introduce site-directed mutations into Fip2 gene in a plasmid encoding kanamycin resistance. The length of the plasmid was about 5 kb. The plasmid was replicated and purified from host cell DH5α which is one of the most popular E. coli strain in laboratories. DH5α expresses methylases constitutively, so that the plasmid was methylated in vivo and the purified plasmid could be used as template for the mutagenesis of the subject invention directly.

### Example 1

The site-directed mutagenesis of Fip2 by using partially complementary primers wherein mutagenesis sites were in the non-complementary region of one primer comprised the steps of:
1. synthesizing two primers which were partially complementary at 5′ end, wherein the mutations were in the non-complementary region for introducing 3-nucleotides substitution which generates an EcoRV cutting site: (substitutions are denoted in capital letters, and the complementary region between forward and reverse primers is underlined)

Primer #1:
cccttgaaaggaaaaattctgGaTAtccatcagag,
and
Primer #2:
cagaatttttcctttcaagggcctgacacttttc;

2. preparing the reaction solution:
   2.5 μl of 10× reaction buffer (BD Biosciences)
      10 ng of Fip2 plasmid (GM Biosciences, Inc)
      0.5 μl (20 μM) of primer #1
      0.5 μl (20 μM) of primer #2
      1 μl of 10 mM dNTPs mix (2.5 mM each dNTP) (BD Biosciences)
      0.5 unit KOD HiFi DNA polymerase (BD Biosciences)
      Double-distilled water to a final volume of 25 μl;
3. incubating the solution of step 2 in a PCR machine (PTC-200 thermocycler, Bio-Rad,) 95° C. for 5 min to denature the template and 18 cycles at 95° C. 30 sec, 60° C. 15 sec, 72° C. 50 sec;
4. transformation of PCR product into ER2925 (New England Biolabs) which was Dam⁻Dcm⁻, comprising:
   a. gently thawing 50 μl of ER2925 competent cell on ice;
   b. adding 2.5 μl of PCR product from step 3 into the competent cell, gently mixing by swirling several times and incubating the mixture on ice for 5 min;
   c. heat shocking the transformation reaction at 42° C. for 30 seconds and then putting the tube on ice for 2 minutes;
   d. adding 250 μL SOC medium, and shaking at 37° C. for 1 hour; and
   e. spreading the entire volume onto a LB plate with kanamycin (50 ng/mL), and culturing overnight at 37° C.

The mutagenesis efficiency was determined by miniprep of colonies grew up and digestion by EcoRV. Expected mutagenesis efficiency was about 90%.

### Example 2

The site-directed mutagenesis of Fip2 by using partially complementary primers wherein mutagenesis sites were in the complementary region of both two primers comprised the steps of:
1. synthesizing two primers which were partially complementary at 5′ end, wherein the mutations were in the complementary region for introducing 3-nucleotide substitution which generates an EcoRV cutting site: (substitutions are denoted in capital letters, and the complementary region between forward and reverse primers is underlined)

Primer #3:
ggaaaaattctgGaTAtccatcagagttgaatgaaaag;
and
Primer #4:
ctctgatggaTAtCcagaatttttcctttcaagggc,

2. preparing the reaction solution:
   2.5 μl of 10× reaction buffer (BD Biosciences)
      10 ng of Fip2 plasmid (GM Biosciences, Inc)
      0.5 μl (20 μM) of primer #3
      0.5 μl (20 μM) of primer #4
      1 μl of 10 mM dNTPs mix (2.5 mM each dNTP) (BD Biosciences)
      0.5 unit KOD HiFi DNA polymerase (BD Biosciences)
      Double-distilled water to a final volume of 25 μl;
3. incubating the solution of step 2 in a PCR machine (PTC-200 thermocycler, Bio-Rad,) 95° C. for 5 min to denature the template and 18 cycles at 95° C. 30 sec, 60° C. 15 sec, 72° C. 50 sec;
4. transformation of PCR product into ER2925 (New England Biolabs) which was Dam⁻Dcm⁻.

The mutagenesis efficiency was determined by miniprep of colonies grew up and digestion by EcoRV. Expected mutagenesis efficiency was about 90%.

### Example 3

Fip2 was mutagenized by two completely complementary primers followed the method of the present invention, which comprised the steps of:
1. synthesizing two primers which were completely complementary, wherein the mutation was for introducing 1-nucleotide substitution: (substitutions are denoted in capital letters)

Primer #5:
gagctcctgaccgCgaaccaccagctgaaag;
and
Primer #6:
ctttcagctggtggttcGcggtcaggagctc;

2. preparing the reaction solution:
   2.5 μl of 10× reaction buffer (BD Biosciences)
      10 ng of Fip2 plasmid (GM Biosciences, Inc).
      0.5 μl (20 μM) of primer #5
      0.5 μl (20 μM) of primer #6
      1 μl of 10 mM dNTPs mix (2.5 mM each dNTP) (BD Biosciences)
      0.5 unit KOD HiFi DNA polymerase (BD Biosciences)
      Double-distilled water to a final volume of 25 μl;
3. incubating the solution of step 2 in a PCR machine (PTC-200 thermocycler, Bio-Rad,) 95° C. for 5 min to denature the template and 17 cycles at 95° C. 30 sec, 60° C. 15 sec, 72° C. 50 sec;
4. transformation of PCR product into ER2925 (New England Biolabs) which was Dam⁻Dcm⁻.

The mutagenesis efficiency was determined by miniprep of colonies grew up and sequencing. Expected mutagenesis efficiency was about 90%.

1. A method for introducing site-directed mutation(s) into a selected circular methylated nucleic acid, comprising:
(a) performing polymerase chain reaction (PCR) using DNA polymerase(s), complementary mutagenic primers, unmethylated dNTPs and a circular methylated nucleic acid to be mutagenized as PCR template;
(b) transforming the mixture of PCR products from step (a) into a methylase deficient E. coli strain, in which mutagenized unmethylated nucleic acids are efficiently replicated; and
(c) recovering the mutagenized unmethylated nucleic acids from the E. coli strain.

2. The method of claim 1, wherein in step (b) the methylase deficiency in methylase deficient E. coli is inducible.

3. The method of claim 1, wherein in step (b) the methylase deficiency in methylase deficient E. coli is non-inducible.

4. The method of claim 1, wherein in step (b) the methylase deficiency in methylase deficient E. coli is permanent.

5. The method of claim 1, wherein in step (b) the methylase deficiency in methylase deficient E. coli is transient.

6. The method of claim 1, wherein the methylase deficient cells are Dam and Dcm deficient E. coli.

7. The method of claim 6, wherein the deficiency of Dam and Dcm is non-inducible and permanent.

8. The method of claim 6, wherein the methylase deficient E. coli strain is strain ER2925 or SK383.

9. The method of claim 1, wherein said circular methylated nucleic acid is methylated in vitro.

10. The method of claim 1, wherein said circular methylated nucleic acid is methylated in vivo.

11. The method of claim 1, wherein in step (a) the primers are partially complementary.

12. The method of claim 1, wherein in step (a) the primers are completely complementary to each other.

13. The method of claim 1, wherein in step (a) the mutagenesis site(s) is in the complementary and/or non-complementary region(s) of the primers.

14. The method of claim 1, wherein in step (a) said DNA polymerase(s) is temperature stable.

15. A kit for introducing mutation(s) into a selected DNA molecule for mutagenesis, said kit comprising: methylase deficient cells.

16. The kit of claim 15, wherein the methylase deficient cells are methylase deficient E. coli.

17. The method of claim 7, wherein the methylase deficient E. coli strain is strain ER2925 or SK383.

18. The method of claim 17, wherein in step (a) the primers are partially or completely complementary to each other.

19. A method for introducing site-directed mutation(s) into a selected circular methylated nucleic acid, comprising:
(a) performing polymerase chain reaction (PCR) using temperature stable DNA polymerase(s), complementary mutagenic primers, unmethylated dNTPs and a circular methylated nucleic acid to be mutagenized as PCR template;
(b) transforming the mixture of PCR products from step (a) into a methylase deficient E. coli strain, in which mutagenized unmethylated nucleic acids are efficiently replicated; and
(c) recovering the mutagenized unmethylated nucleic acids from the E. coli strain;
wherein the methylase deficient cells are Dam and Dcm deficient E. coli, the deficiency of Dam and Dcm being non-inducible and permanent, and the methylase deficient E. coli strain is strain ER2925 or SK383.

20. The method of claim 19, wherein in step (a) the primers are partially or completely complementary to each other.
